(19) United States
(12) Patent Application Publication
(10) Pub. No.: US 2010/0297639 A1
(43) Pub. Date: Nov. 25, 2010

(54) QUANTITATIVE/SEMI-QUANTITATIVE MEASUREMENT OF EPOR ON CANCER CELLS

(75) Inventors: Christopher P. Miller, Seattle, WA (US); Carl Anthony Blau, Seattle, WA (US); Michael Henke, Freiberg (DE)

Correspondence Address: NIXON PEABODY LLP - PATENT GROUP, 1100 CLINTON SQUARE, ROCHESTER, NY, 14604, US

(73) Assignee: UNIVERSITY OF WASHINGTON through its Center for Commercialization, Seattle, WA (US)

(21) Appl. No.: 12/682,105

(22) PCT Filed: Oct. 8, 2008
(86) PCT No.: PCT/US08/79128

§ 371 Date: Jul. 8, 2010

Related U.S. Application Data
(60) Provisional application No. 60/960,639, filed on Oct. 9, 2007.
(60) Provisional application No. 61/037,790, filed on Mar. 19, 2008.

(51) Int. Cl. C12Q 1/68; G01N 33/53; G01N 33/574

(52) U.S. Cl. 435/6; 435/7.92; 435/7.23

(57) ABSTRACT

The present invention provides for assays useful in predicting whether a cancer patient risks suffering erythropoietin-induced tumor progression if treated with erythropoietin. More specifically, one embodiment provides for a validated quantitative reverse transcriptase polymerase chain reaction assay that detects erythropoietin receptor expression, thus indicating whether a cancer patient risks suffering erythropoietin-induced tumor progression if treated with erythropoietin.

### RELATED APPLICATIONS

This application claims priority from U.S. Provisional Patent Applications No. 61/960,639, filed Oct. 9, 2007; and No. 61/037,790, filed Mar. 19, 2008; each entitled Quantitative/Semi-quantitative Measurement of EpoR on Cancer Cells, by Christopher P. Miller, C. Anthony Blau, and Michael Henke.

This invention was made with government support under grants No. R01DK 74522-01 and No. P01 HL53750-02 awarded by the National Institutes of Health, and contract No. CDMRP, DAMD17-02-0691 awarded by the Department of Defense. The government has certain rights in the invention.

### FIELD OF THE INVENTION

The present invention relates to molecular genetics, molecular immunology, and cancer diagnostics.

### BACKGROUND

Recombinant erythropoietin (Epo) is an erythropoiesis-stimulating agent (ESA) used widely for the treatment of cancer-related anemia. Several studies suggest, however, that erythropoietin may exert unanticipated negative effects in cancer patients. One explanation for the possible adverse effects of Epo in cancer may reside in the finding that some non-hematopoietic cells carry Epo receptors (EpoRs). Several reports suggest that many cancer cell types use the Epo system for growth and angiogenesis and indeed, it now appears that Epo may promote tumor progression. The FDA has recognized the potential danger associated with Epo treatment, and issued a “black box” safety warning regarding Epo use in cancer patients.

There is currently no way of knowing whether a given patient is at risk for Epo-induced tumor progression. Accordingly, it would be desirable to have improved methods to diagnose which patients may be impacted adversely by Epo administration. In particular, there is a need for superior diagnostics that may determine whether EpoR is expressed by a cancer patient's tumor.

### SUMMARY

The present invention provides for methods for determining the risk that a cancer patient treated with erythropoietin (Epo) or erythropoiesis-stimulating agents (ESAs) may experience tumor progression due to Epo or ESA treatment.

In one embodiment of the invention, the risk is determined by obtaining a tumor sample from the patient, contacting the sample with Epo, and detecting the presence of Epo bound to Epo receptor (EpoR), wherein increased detection of Epo in the sample, as compared to control-levels, indicates increased risk of tumor progression.

Another embodiment provides for a method for determining the risk that a cancer patient treated with an ESA may experience tumor progression due to the ESA treatment by obtaining a tumor sample from the patient, and detecting EpoR, Jak2, and/or Hsp70 mRNA in said sample, wherein level of expression of one or more of these biomarkers in said sample, as compared to control-level expression, indicates increased risk of tumor progression if the patient is treated with an ESA.

Yet another embodiment of the invention is a method for evaluating the risk that a cancer patient treated with an ESA may experience tumor progression due to ESA treatment comprising the steps of determining whether the patient has undergone tumor resection, and determining the level of EpoR, Jak2, and/or Hsp70 expressed in the patient's tumor, wherein a patient with an unresected tumor that expresses above the median EpoR and/or Jak2 mRNA, or below the median Hsp70 mRNA, is at risk for tumor progression if treated with an ESA.

Another embodiment provides for a method for diagnosing whether a human subject having cancer should be treated with an ESA by obtaining a tumor sample from the subject, obtaining RNA from the sample, performing quantitative reverse transcriptase-polymerase chain reaction (RT-PCR) on the RNA using primers that amplify a EpoR mRNA, Jak2 mRNA, and/or Hsp70 mRNA in the sample; then comparing the amount of EpoR, Jak2, and/or Hsp70 mRNA amplification product with the amount of EpoR, Jak2, and/or Hsp70 mRNA amplification product in a control sample, wherein an increase in the amount of EpoR and/or Jak2 mRNA, and/or a decrease in the amount of Hsp70 mRNA amplification product in the tumor of the subject, as compared to the amount of EpoR, Jak2, and/or Hsp70 mRNA amplification product in the control sample, indicates that the subject may suffer tumor progression if treated with Epo or an ESA.

Still another embodiment provide for a method for determining the risk that a cancer patient treated with Epo may experience tumor progression due to Epo treatment by obtaining a tumor sample from said patient, contacting said sample with antibody against EpoR, detecting the presence of EpoR, wherein increased detection of EpoR in said sample as compared to control-levels is indicative of increased risk of tumor progression.

Another embodiment of the present invention provides for a diagnosis kit for detecting EpoR, Jak2, and Hsp70 mRNA expression in a tumor, including at least one container means for accepting a tumor sample, primers for EpoR, Jak2, and Hsp70 mRNA, and at least one reagent for carrying out PCR amplification.

### DESCRIPTION OF THE DRAWINGS

FIGS. 1A-1D present data demonstrating that EpoR mRNA levels can distinguish Epo-responsive from non-responsive cancer cell lines, and can be characterized in archival formalin-fixed, paraffin-embedded tumors. FIG. 1A, Top Panel, shows EpoR mRNA levels measured by quantitative RT-PCR using RNA extracted from the indicated cell lines. The 769P cells do not express EpoR (Elliott et al., 107(5) Blood, 1892-95 (2006)), and served as a negative control. EpoR mRNA levels normalized to the endogenous control genes, hydroxymethylbilane synthase (Hmbs) or polymerase RNA II DNA directed polypeptide A (Polr2a), in each cell line are shown. Similar results were obtained upon normalization to polymerase RNA II DNA directed polypeptide A (Polr2a) or transferrin receptor (Tfrc). Values are expressed as a percentage of the EpoR mRNA level in erythroid ASE2 cells (these erythroid cells are defined in Inoue et al., 34(1) Exp Hematol. 19-26 (2006). Error bars represent the standard error of measurements obtained from 4 independent RNA extractions from separate culture passages. FIG. 1A, Bottom Panel, shows data from cells stained with a murine monoclonal anti-EpoR phycoerythrin (PE) conjugated antibody and analyzed by flow cytometry. isotype control, mIgG2b-PE.

FIG. 1B provides data from murine Ba/F3 and Ba/F3-hEpoR cells that were combined in the indicated ratios, fixed in formalin, and embedded in paraffin prior to RNA extraction. EpoR levels were normalized to the murine endogenous control gene phosphoglycerate kinase 1 and are expressed relative to the level in control Ba/F3 cells. Error bars represent the standard deviation of triplicate PCR determinations.

FIG. 1C reflects EpoR mRNA levels normalized to Hmbs, shown in formalin-fixed paraffin-embedded and snap frozen fragments from the same breast tumor.

FIG. 1D shows EpoR mRNA levels normalized to Hmbs in each of 101 head and neck cancer cases from the ENHANCE clinical trial. To demonstrate that normalization effectively corrected for differences in RNA abundance/integrity, tumors are arranged in order of low to high RNA abundance/integrity as determined by PCR cycle threshold values for the control gene Hmbs. Five samples with EpoR mRNA levels of >1 standard deviation from the mean are indicated by the hash marks below the x-axis. The coefficient of variance of triplicate PCR determinations was <4% for all assays.

FIGS. 2A-2D show the analysis of the effects of exogenous and endogenous Epo on clinical outcome with stratification by marker status. FIG. 2A presents patients stratified by EpoR mRNA status as above versus below/equal to the median expression value. The proportion of patients at risk for tumor progression or death is shown over time by Kaplan Meier curves, and the significance of differences in outcomes in response to Epo versus placebo were evaluated with the log rank test. P values are two-sided. Analyses are shown for all patients and in the subgroup of patients undergoing definitive radiotherapy with no tumor resection. The log rank p value for direct comparison of outcomes of Epo-treated patients in the no resection stratum expressing above versus below median EpoR mRNA is indexed below the bracket.

FIG. 2B shows data for Hsp70 mRNA. FIG. 2C shows data for Jak2 mRNA. FIG. 2D presents patients stratified by C20 staining status as positive or negative. The log rank test was used to evaluate the significance of differences between outcomes in response to high baseline serum Epo levels versus low baseline Epo levels, where ≦11 U/L was defined as low and >11 U/L was defined as high. Analyses are shown for all patients and in the subgroup of patients with residual tumor following surgery (incomplete resection plus no resection groups).

FIGS. 3A and 3B show the relationship between C20 staining results and mRNA levels for EpoR and for heat shock protein 70 (Hsp70). FIG. 3A is EpoR mRNA levels normalized to Hmbs in each of 100 tumors, shown in comparison to the results of tumor characterization with the C20 rabbit polyclonal anti-EpoR antibody by immunohistochemistry. C20 data was not available for one tumor for which EpoR mRNA data was available. Spearman's correlation coefficients are indexed. No trend was observed. FIG. 3B shows levels of mRNA for each member of the Hsp70 family normalized to Hmbs are shown for each tumor compared with C20 status. A trend in which the highest Hsp70 mRNA expressors tended to fall in the C20 positive category was not statistically significant.

FIG. 4 reflects limited utility of EpoR protein detection in cancer cells using EpoR-specific antibodies by immunohistochemistry. Formalin-fixed, paraffin-embedded sections (6 micron) were stained using goat polyclonal anti-human EpoR (ab10653, Abcam) and biotinylated anti-goat antibodies. Staining was visualized using the Vector Elite ABC system and 3,3′-diaminobenzidine. Sections were counterstained with hematoxylin. Negative controls included Ba/F3, Cos, and 769P cells, while positive controls included Ba/F3-hEpoR, Cos-hEpoR, and ASE2 cells.

FIG. 5 illustrates concordance in EpoR measurements between snap-frozen and formalin-fixed paraffin-embedded breast tumors. mRNA levels of each of the indicated genes were normalized to Hmbs and the rank order of expression among twenty-three breast tumors is plotted. Results were obtained using RNA extracted from snap frozen (y-axis) versus FFPE (x-axis) pieces of the same breast tumor. Estrogen receptor (Esr1), a known prognostic factor in breast cancer, was used as a positive control. The coefficient of variance of triplicate PCR determinations was <4% for all assays. Spearman's rank order correlation coefficients are shown above each graph.

FIG. 6 shows effects of RNA abundance/integrity on normalized EpoR relative quantification values. EpoR mRNA levels normalized to peptidylprolyl isomerase A (Ppia) are shown in each of 106 head and neck cancer tumors. Tumors are arranged in order of low to high RNA abundance/integrity to demonstrate the influence of RNA abundance/integrity on normalization. Less RNA abundance/integrity was associated with higher relative EpoR quantification upon normalization with Ppia. This systematic effect was corrected upon normalization to Hmbs, which had the shortest amplicon size among all reference gene assays tested (64 bp) (see FIG. 1D). The coefficient of variance of triplicate PCR determinations was <4% for all assays.

FIG. 7 is Table 1, identifying the genes analyzed throughout this application.

FIG. 8 presents Table 2, listing control genes ranked in order of increasing stability from top to bottom according to the GeNorm algorithm (Vandesompele et al., 3(7) Genome Biol. r0034.1-r0034.11 (2002).

FIG. 9 is Table 3, with data showing that preamplification uniformly decreases cycle threshold values without biasing relative quantification values.

FIG. 10 presents Table 4, reflecting the analysis of exogenous Epo administration and clinical endpoint by mRNA marker status.

FIG. 11 is Table 5, the analysis of endogenous baseline Epo and clinical endpoint in placebo treated patients by marker status.

### DETAILED DESCRIPTION

It should be understood that this invention is not limited to the particular methodology, protocols, and reagents, etc., described herein and as such may vary. The terminology used herein is for the purpose of describing particular embodiments only, and is not intended to limit the scope of the present invention, which is defined solely by the claims.

As used herein and in the claims, the singular forms include the plural reference and visa versa unless the context clearly indicates otherwise. Other than in the operating examples, or where otherwise indicated, all numbers expressing quantities of ingredients or reaction conditions used herein should be understood as modified in all instances by the term “about.”Unless defined otherwise, all technical and scientific terms used herein have the same meaning as those commonly understood to one of ordinary skill in the art to which this invention pertains.

All patents and other publications identified are incorporated herein by reference for the purpose of describing and disclosing, for example, the methodologies described in such publications that might be used in connection with the present invention, but are not to provide definitions of terms inconsistent with those presented herein. These publications are provided solely for their disclosure prior to the filing date of the present application. Nothing in this regard should be construed as an admission that the inventors are not entitled to antedate such disclosure by virtue of prior invention or for any other reason.

Erythropoietin (Epo) is a glycoprotein hormone produced in the kidney, responsible for the regulation of RBC production. Epo receptor (EpoR) is a receptor found on the surface of erythroid progenitor cells in the bone marrow that when bound with Epo, stimulates erythroid progenitor cells to transform into erythrocytes. By binding to Epo receptors on erythroid progenitor cells, Epo is responsible for progenitors maturing to functional erythrocytes. Recombinant erythropoietin is therapeutically administered to patients who develop anemia.

Anemia has been implicated as an independent poor prognostic factor for survival in patients with cancer. Caro et al., 91(12) Cancer, 2214-21 (2001). Because anemia is common in cancer patients, recombinant Epo has become a mainstay in oncology, raising hematocrits and reducing transfusion requirements. Recent phase III studies testing off-label use suggest, however, that Epo shortens cancer survival times due partly to thrombotic events but due primarily to accelerated tumor progression. Henke et al., 362(9392) Lancet, 1255-60 (2003); Leyland-Jones et al., 23(25) J. Clin. Oncol. 5960-72 (2005); Wright et al., 25(9) J. Clin. Oncol. 1027-32 (2007); Smith et al., 26(7) J. Clin. Oncol. 1040-50 (2008); Bennett et al., 299(8) JAMA, 914-24 (2008). The first of these studies (“ENHANCE”) was a randomized multi-center trial involving 351 patients with head and neck cancer randomized to Epo or placebo concomitant with radiation therapy following complete resection, partial resection, or no resection of their tumors. Henke et al., 2003.

The embodiments presented herein provide for diagnostic test for predicting the risk of Epo-induced tumor progression. This provides an important resource for doctors and their patients, because Epo is used widely in patients with cancer, and has been implicated recently in promoting tumor progression. There is currently no way of knowing whether a given patient is at risk for Epo-induced tumor progression. With the methods provided for herein, one can test a piece of tumor tissue at the time of diagnosis (or subsequently) to assess the risk for tumor progression related to Epo treatment. Additionally, patients susceptible to ESA-induced tumor growth may be identified prospectively.

The predictive power of the present invention has been validated using tumor samples from patients who were enrolled in a Phase III trial testing whether Epo treatment influenced survival in cancer patients. The present methodology was optimized for use with formalin-fixed paraffin embedded tumors. The control gene to which EpoR was compared across different tumors was selected carefully by comparing endogenous controls that vary minimally across a number of different cancers of the breast, neck, and head. Because of the low quantity of mRNA and its highly degraded nature, experimentation and innovation was required to determine the relative abundance of EpoR mRNA across different tumors.

Previous studies have demonstrated that only a tiny fraction of EpoR protein reaches the cell surface, with the majority remaining in the cytoplasm. This raised the expectation that the amount of EpoR protein on the cell surface, and trafficking of EpoR from the cytoplasm to the membrane are more likely to be critical for determining EpoR's biological activity in tumors. That EpoR mRNA levels hold prognostic significance, as demonstrated herein, is therefore surprising and unexpected. Moreover, previous studies suggested that Epo's ability to stimulate EpoR in non-erythroid tissues depends on the presence of a subunit shared by several different receptors called colony stimulating factor 2 receptor beta or “the common beta chain” (βcR). Brines et al., 101(41) P.N.A.S. USA 14907-12 (2004). That EpoR expression would be of diagnostic importance independent of expression of βcR is also unexpected.

Moreover, quantitative RT-PCR provides an improvement over the pre-existing polyclonal antibody techniques. Real-time or kinetic PCR is a powerful method for determining the initial template copy number. The quantitative information in a PCR reaction comes from the few cycles where the amount of DNA grows logarithmically from barely above background to the plateau. Often only six to eight cycles out of forty will fall in this log-linear portion of the curve. Because the fluorescence signal is acquired during each cycle, data from the critical cycles can be captured, quantified and the fluorescence plotted against the cycle number.

Concern that a widely used drug in oncology might surreptitiously accelerate cancer progression and death has generated intense scrutiny by the FDA and the Centers for Medicare and Medicaid Services. A black box warning for Epo, issued in March 2007 was strengthened in November 2007. Wide discordance exists regarding how oncologists and regulators should respond, however. Integral to this uncertainty is a lack of knowledge regarding how Epo might promote cancer progression or whether this can be predicted. Critical to overcoming this uncertainty are methods that will allow us to understand whether the adverse effects of Epo observed in multiple clinical trials were an indirect consequence of Epo action on erythroid cells, were due to unrecognized thrombotic events, or mainly reflect “off-target” effects of Epo on tumor cells and/or tumor blood vessels.

The invention described herein provides for key insights into Epo-induced tumor progression that may reside in archival tumors from subjects enrolled in completed phase III trials. Efforts to examine tumor EpoR protein levels in archival tumors have been hampered by the lack of specificity of commercial antibodies, and low-level EpoR expression. A method for overcoming this limitation by characterizing EpoR mRNA levels in formalin-fixed paraffin-embedded tumors is described herein. A set of head and neck cancers previously evaluated using the C20 antibody, revealed a wide (>30 fold) range of EpoR mRNA expression and, in the subset of patients undergoing primary radiotherapy with no tumor resection, that above-median EpoR transcript levels were associated with a worse outcome in patients randomized to Epo compared to placebo. In contrast, no association was found between above-median EpoR mRNA level, Epo treatment, and outcome in patients with completely or partially resected tumors. Thus, apparent adverse effects of EpoR mRNA seemed limited to the group of patients with the most remaining residual tumor. A similar relationship was identified for Jak2, the intracellular signal transducing component of the EpoR. These results may be interpreted with caution, however. When locoregional progression-free survival analysis was confined only to Epo-treated subjects with above-median versus below-median EpoR, a trend towards unfavorable locoregional progression-free survival in patients with above-median EpoR mRNA levels did not reach statistical significance. Nonetheless, EpoR and Jak2 transcript levels join C20 staining as a candidate predictive test for Epo-associated tumor progression. In keeping with C20's documented lack of specificity for EpoR, there was no correlation between C20 status and EpoR transcript levels.

Measuring the influence of baseline Epo levels on outcomes in patients randomized to the placebo group provided a second look at the influence of C20 staining and EpoR transcript level on outcome. Employing a cut-off baseline Epo level of 11 was reasonable based on a previous report showing that Epo levels of >10.5 are associated with an increased risk of recurrence in surgically resected patients with non-small cell lung cancer. Paul et al., 51(3) Lung Cancer 329-34 (2006). Remarkably, the present invention showed that an elevated baseline Epo level was associated with a significantly poorer locoregional progression-free survival in patients with C20 positive tumors, while trending toward a better locoregional progression-free survival in patients with C20 negative tumors.

This novel finding supports the previously published report showing that C20 staining may identify patients susceptible to tumor progression when administered exogenous Epo (Henke et al., 24(29) J. Clin. Oncol. 4708-13 (2006)) and, in the context of the finding that EpoR mRNA and C20-staining do not correlate, may indicate that additional molecular markers may predict tumor progression in response to Epo.

In light of recent studies showing that C20 cross-reacts with HSP70 family members (Elliott et al., 2006), and that Epo regulates the ability of HSP70 to protect erythroid cells from caspase-mediated transcription factor degradation (Ribeil et al., 445 Nature, 102-05 (2007)), mRNA levels for each of the eight Hsp70 family members were measured. Although there was a trend toward higher Hsp70 levels in C20 positive tumors, this was not significant. Moreover, none of these markers, alone or in aggregate, mirrored C20's apparent predictive value for adverse outcome in response to Epo. To the contrary, below median levels of Hsp70 family members correlated with a poorer outcome in non-resected patients treated with Epo. These unexpected findings may hold important biological meaning.

It should be noted, however, that limited material precluded measurements of EpoR transcript levels in all patients. In the ENHANCE study, statistically significant adverse effects of Epo treatment were confined to patients with incompletely resected or unresected tumors, and RNA yields prevented determinations of EpoR mRNA levels for a significant fraction of patients in the unresected group. These findings may also be constrained by lack of access to additional tumors from other phase III clinical trials of Epo in cancer. Because these were large multicenter trials lacking centralized tumor repositories, obstacles to obtaining these tumors can likely only be surmounted by the trial sponsors.

Nevertheless, the findings presented herein provide for critical methodology and point to hypotheses to be tested in tumors from additional phase III trials. If upheld, the clinical implications related to Epo in cancer would be substantial. Of most relevance is the availability of a predictive test to identify tumors susceptible to Epo induced growth. If the correlation between C20 status and susceptibility to tumor progression in the setting of elevated endogenous Epo levels is consistent, it would indicate that Epo induced tumor progression is not confined to exogenous Epo administration, but can also occur at Epo levels achieved endogenously. Finally, confirmation of a role for Epo in tumor progression could point to a new way of treating cancer by blocking Epo signaling, as suggested by a recent preclinical study. Hardee et al., 2(6) PLoS ONE:e549 (2007).

### EXAMPLES

### Example 1

### Cancer Cell Lines and Archival Breast Tumors Can Be Reliably Characterized for EpoR mRNA Expression

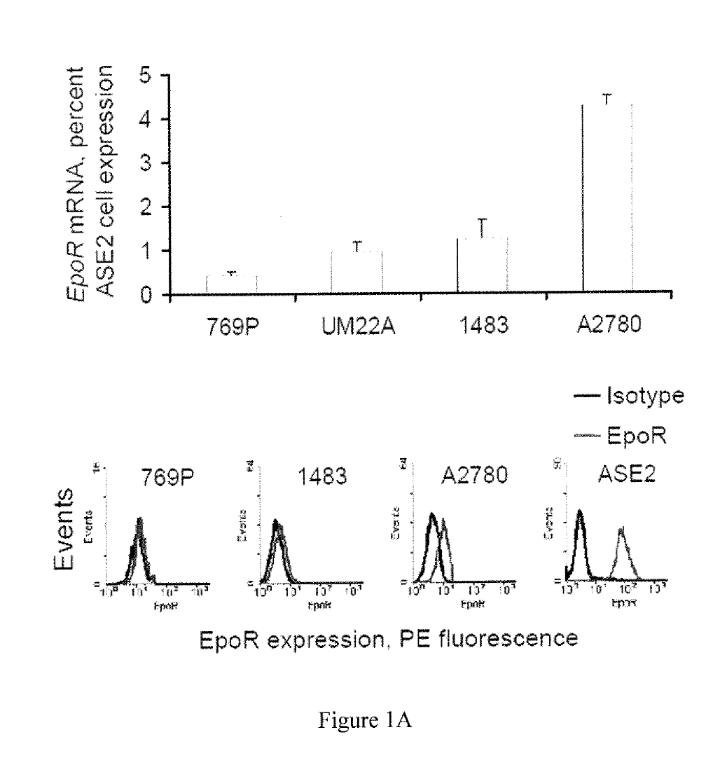

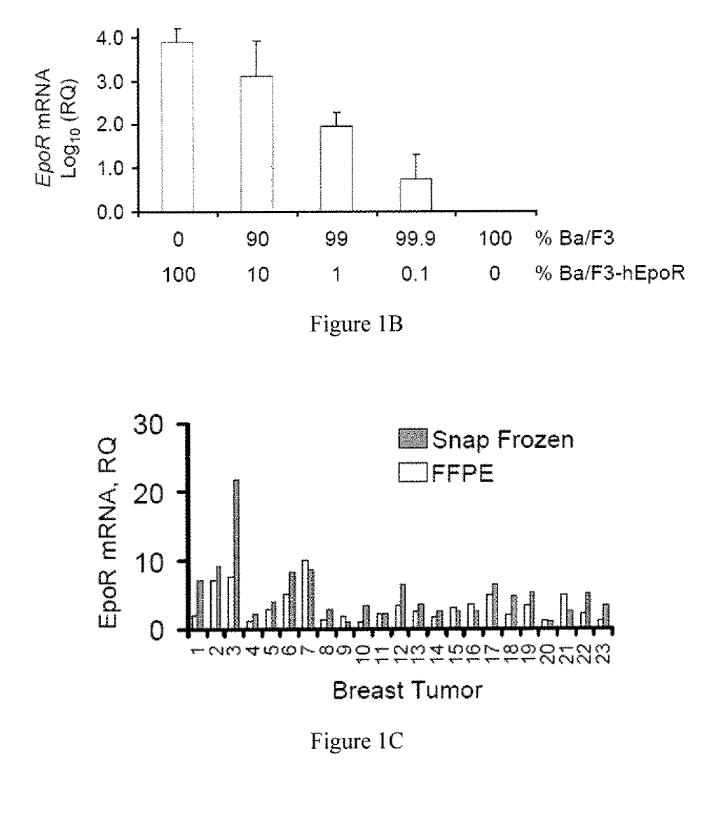

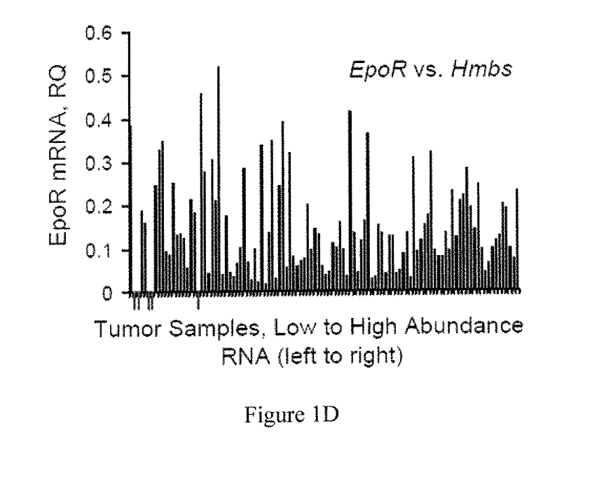

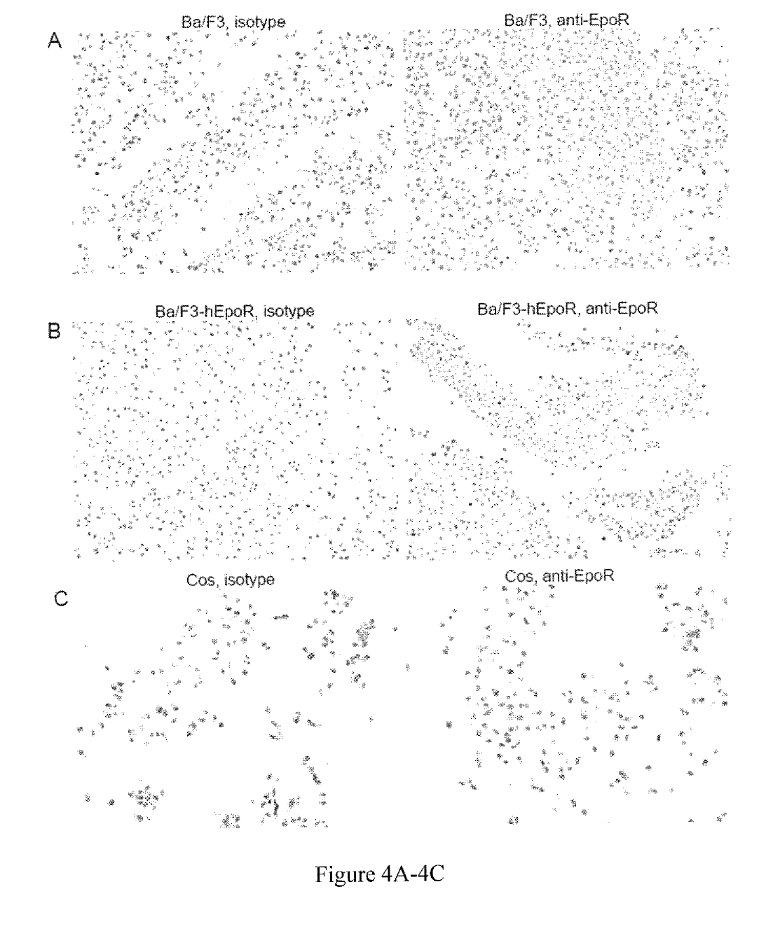

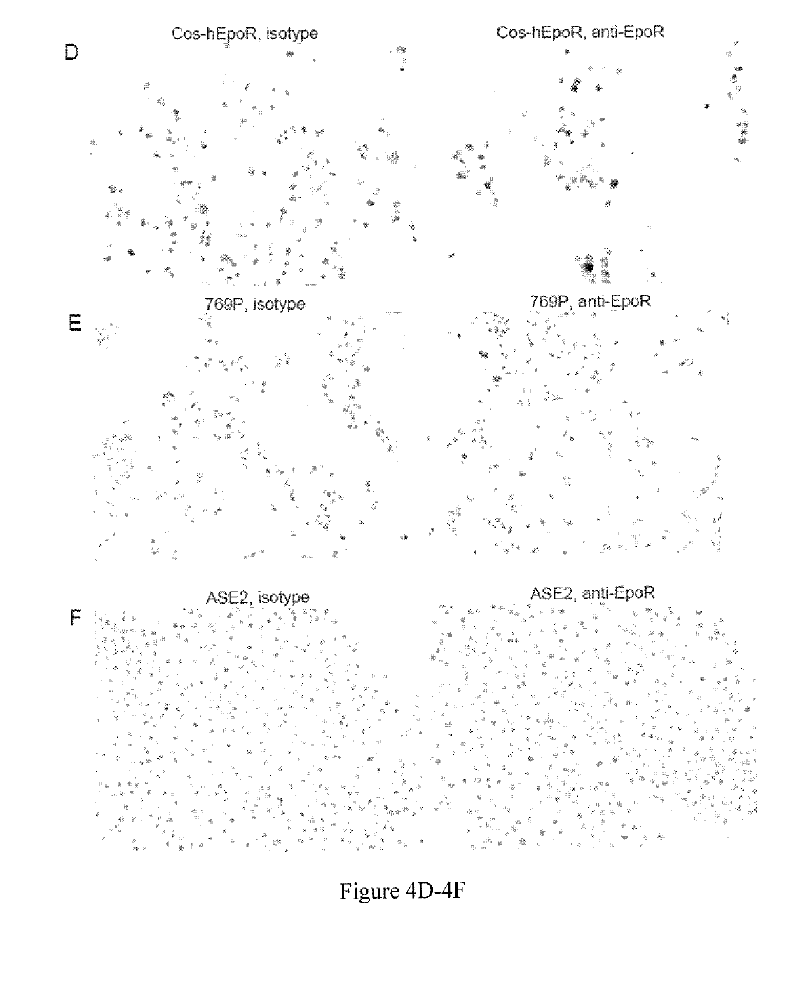

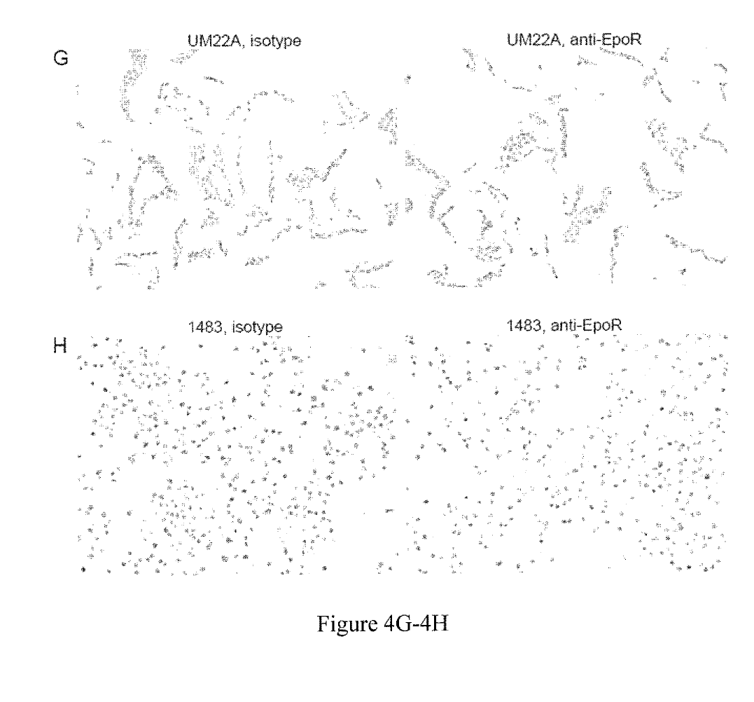

Importantly, the diagnostic methods of the present invention may be applied to archival clinical trial samples. In a clinical setting, there may be limited utility of EpoR protein detection in tumor cells using current EpoR-specific antibodies by immunohistochemistry. Reports indicate that several commonly used EpoR antibodies lack suitable specificity for EpoR detection by immunohistochemistry. In addition, immunohistochemistry with specific antibodies was found to be insufficiently sensitive to detect EpoR protein in tumor cell lines and primary tumors (FIG. 4). In contrast, quantitative RT-PCR measured higher levels of EpoR mRNA in three cancer cell lines previously reported to express functional EpoR (Lai et al., 24(27) Oncogene, 4442-49 (2005); Solar et al., 122(2) Int'l J. Cancer, 281-88 (2008)) than in 769P cells which have previously been used a negative control (Elliott et al., 2006) (FIG. 1A, Top Panel). The identification of all genes analyzed here and throughout this application are provided in Table 1. Notably, although A2780 ovarian cancer which express detectable cell surface EpoR protein (FIG. 1A, Bottom Panel) expressed the highest level of EpoR mRNA, this amounted to less than 5% of the EpoR mRNA level present in erythroid ASE2 cells (these cells are described in Inoue et al., 2006).

The majority of tumors available from clinical trials are preserved as formalin-fixed paraffin-embedded tissue, and formalin-fixed paraffin-embedded tumor-derived RNA is highly degraded. Protocols for measuring EpoR levels using RNA extracted from formalin-fixed paraffin-embedded tissue were tested. RNA was extracted from formalin-fixed paraffin-embedded tumor sections using the Absolutely RNA® extraction kit (Stratagene, La Jolla, Calif.) with deoxyribonuclease I digestion to remove genomic DNA. First strand, complementary DNA (cDNA) was synthesized with random primers and Superscript® III reverse transcriptase (RT) (Invitrogen, Carlsbad, Calif.), which was omitted for no-RT control reactions. cDNA targets were amplified using the TaqMan® gene expression system and a 7900HT thermal cycler (Applied Biosystems, Foster City, Calif.). With the exception of certain intronless members of the Hsp70 family, all probes recognized exon junctions to prevent genomic DNA amplification. Preamplification of cDNA was performed with the TaqMan® preamplification multiplex system (Applied Biosystems). Cycle threshold (Ct) values were determined with the Sequence Detection Software (Applied Biosystems). A coefficient of variance <4% for triplicate Ct determinations was considered acceptable. Relative quantification was determined using the comparative Ct method, 2̂^{-ΔCT} where the difference in (delta, Δ) Ct=mean Ct for target gene−mean Ct for reference gene.

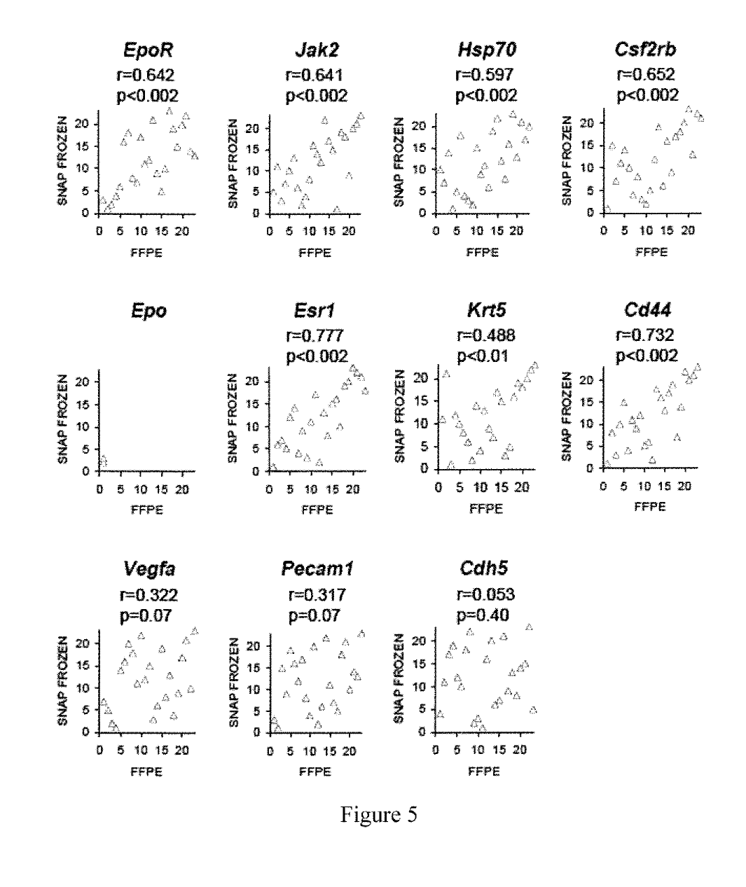

Normalized EpoR expression levels in idealized formalin-fixed paraffin-embedded tissues comprised of defined mixtures of EpoR+ and EpoR− cell lines are shown in FIG. 1B. The accuracy of EpoR mRNA measurements from formalin-fixed paraffin-embedded primary tumors was tested by comparing results obtained with higher quality mRNA extracted from the same tumors stored as snap-frozen tissue, using an established breast cancer repository. Because the formalin-fixed paraffin-embedded and snap-frozen samples represent different pieces of the same tumor, and snap-freezing preserves a higher degree of RNA integrity, this comparison allowed the simultaneous assessment of potential artifacts arising from RNA degradation due to formalin-fixation and the uniformity with which the various markers are expressed across tumors. Normalized EpoR relative quantification values in twenty-three breast tumors stored both as formalin-fixed paraffin-embedded and snap-frozen tissue were highly correlated (r=0.642, p<0.002, n=23) (FIGS. 1C and 5).

The mRNA levels were measured for Jak2 and Hsp70, which participate in Epo signaling in erythroid cells; Csf2rb, which has been suggested to enhance Epo signaling in non-erythroid cells, endothelial-associated genes (Cdh5, Pecam1 Vegfa); the squamous epithelial marker Krt5; the putative cancer stem cell marker Cd44; and Epo itself. Significant correlations between formalin-fixed paraffin-embedded and snap-frozen mRNA measurements were observed for EpoR, Csf2rb, Jak2, Hsp70, Cd44, Krt5 and Esr1 (estrogen receptor-1, used as a positive control) (FIG. 5). In contrast, Vegfa, Cdh5, and Pecam1 were not significantly correlated, consistent with regional heterogeneity in tumor vascularity while Epo was detected in too few formalin-fixed paraffin-embedded tumors to permit calculation of a correlation coefficient.

### Example 2

### EpoR mRNA Expression in Head and Neck Cancers from the ENHANCE Study

The assay described in Example 1 was applied to tumors from a previously reported Phase III trial. Patients in a multi-center, randomized, double-blind, placebo-controlled trial who were treated at the University of Freiburg were included. The trial was approved by the local ethics committee and conducted in accordance with the revised Declaration of Helsinki and good clinical practice guidelines. The Institutional Review Board of the University of Washington approved the analysis of these tumors. Patient selection, treatment, follow-up, evaluation, and baseline characteristics were described (Henke et al., 2003). Briefly, the main inclusion criteria were squamous cell carcinomas of the head and neck with T3 or T4 tumors or nodal involvement, scheduled definitive or postoperative radiotherapy, and a decreased blood hemoglobin (<13 g/dL men; <12 g/dL women) at randomization. Patients were randomly assigned to 300 international units/kg epoetin beta or placebo three times per week starting 10 days to 14 days before radiotherapy, continuing throughout.

Prior to randomization, patients were stratified by resection status, (1) complete resection; (2) incomplete resection; or (3) unresected disease. Iron (III) saccharate (200 mg) was administered intravenously once weekly to patients with <25% transferrin saturation. Epoetin beta was stopped if hemoglobin increased more than 2 g/dL within 1 week or when targets were reached (≧15 g/dL men; ≧14 g/dL women) and continued when hemoglobin fell below target. Locoregional cancer control and survival was assessed at 3-month intervals by an independent oncologist under double-blinded conditions. The primary endpoint was locoregional progression free survival, the time to locoregional tumor progression or death. Tumor progression was noted if the tumor recurred or increased by 25%. Baseline serum Epo levels were determined prior to treatment. Retrospective staining with the polyclonal rabbit anti-human EpoR C20 antibody (Santa Cruz Biotechnology, Santa Cruz, Calif.) was described in Henke et al., 2006.

In quantitative RT-PCR assays of samples from the ENHANCE trial using custom TaqMan® low density arrays, seven candidate reference genes were included for normalization. The gene symbols for these reference genes are Hprt1, Ppia, Ipo8, Hmbs, Gapdh, Tfrc, and Rplp0. These reference genes were included based on their stability, among sixteen candidates tested using the geNorm algorithm (Vandesompele et al., 3(7) Genome Biol. research 0034.1-11 (2002)), in a panel of eight breast cancers or head and neck cancers, as shown in Table 2.

Based on these stability rankings, these control genes were selected to allow this array to be used for both breast cancer and head and neck cancer samples. Therefore, not all control genes were optimal for both cancer types. Results for Hprt were excluded due to high or no Ct values in many samples. For the samples with sufficient RNA (representing 123 different tumors), there were strong positive correlations in Ct values among all reference genes (r≧0.88 for all pairwise comparisons, all highly significant p<0.001).

Because most head and neck cancer samples from the ENHANCE trial consisted of only a single microscope slide with minimal tissue, 14 cycles of target-specific preamplification for all genes using the TaqMan® preamplification multiplex system (Applied Biosystems) were employed. Preamplification uniformity (lack of bias) was confirmed by comparing relative quantification values obtained with unamplified cDNA versus those obtained with preamplified cDNA. This was performed using several tumor samples that contained sufficient RNA, non-limiting erythroid EpoR+ ASE2 cells, and a universal human total RNA standard (Stratagene) so that results obtained with both unamplified and preamplified cDNA could be compared. The difference in cycle threshold (ΔCt) values for data obtained with both unamplified and preamplified cDNA, were calculated, where ΔCt=mean Ct for target gene−mean Ct for reference gene. Uniformity comparisons for all genes (ΔCt preamplified−ΔCt unamplified) were within the acceptable tolerance of variation of 1.5 cycles (ABI). Representative results including EpoR, Hmbs, and the Hsp70 family members using a universal human total RNA standard are shown in Table 3.

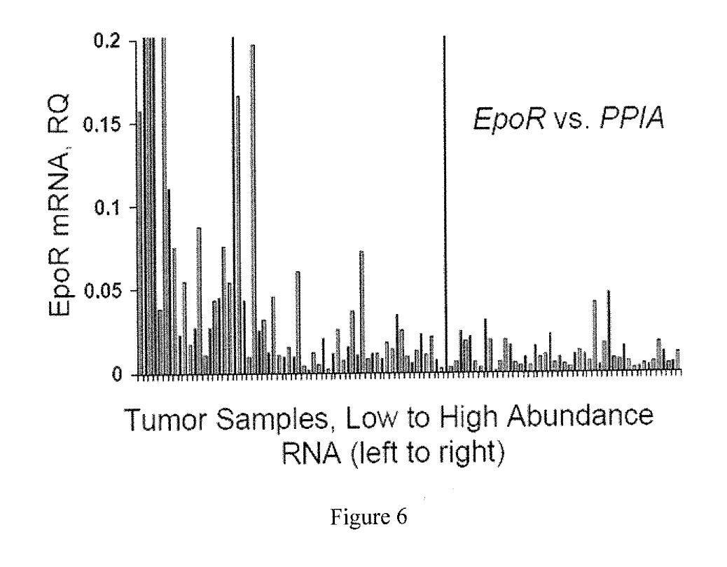

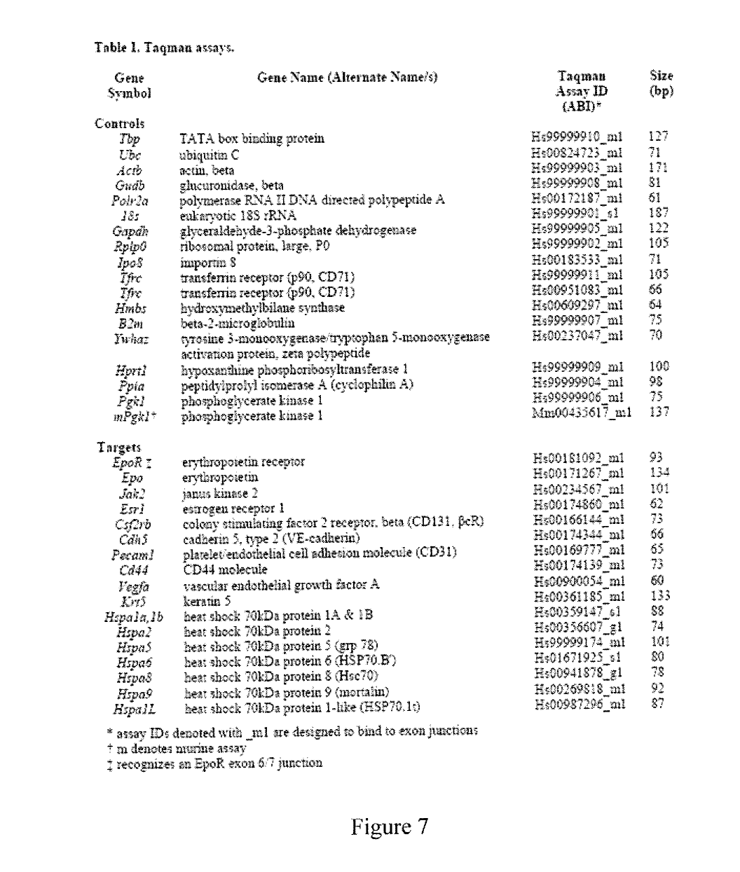

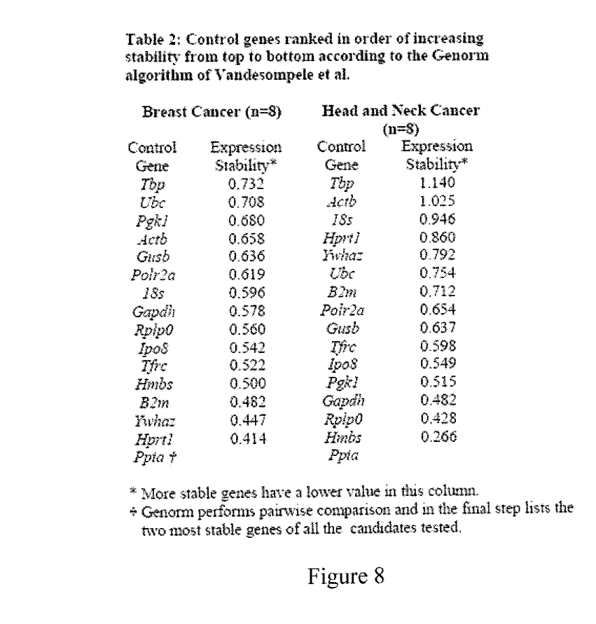

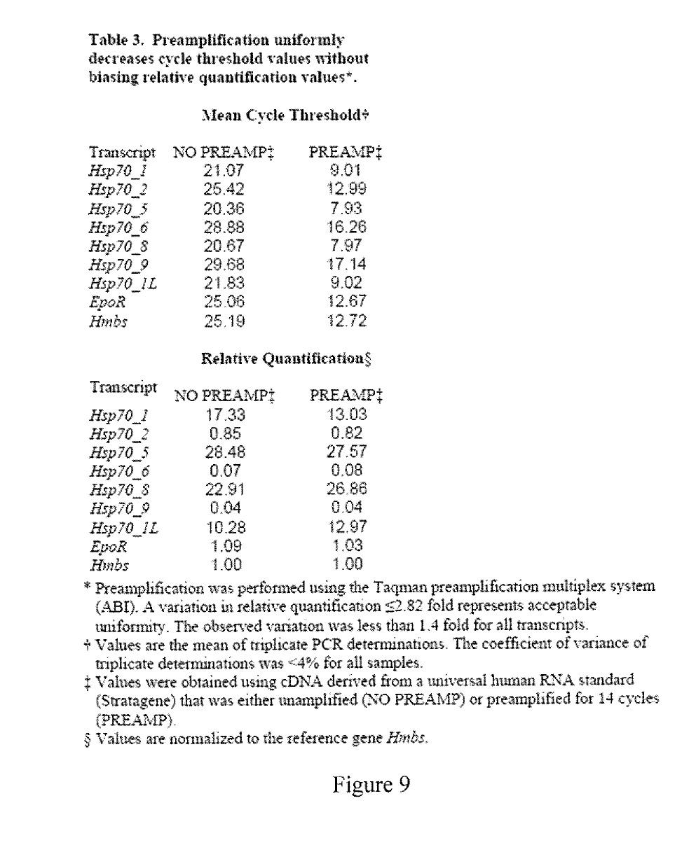

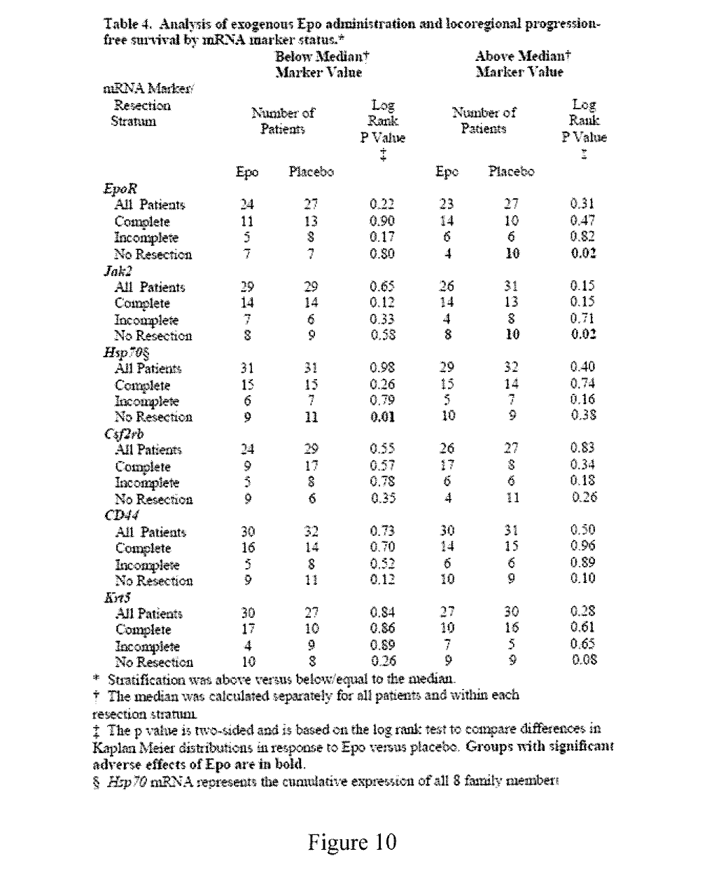

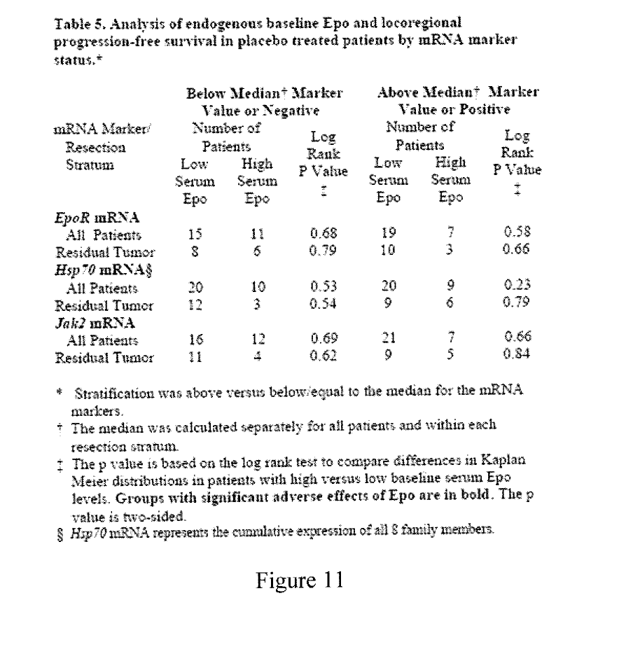

For thirty tumors from the ENHANCE trial, Ct values for EpoR were undetectable and this was often associated with high or undetectable Ct values for the reference genes, indicating that insufficient RNA was available or that the RNA degradation was too extensive. Using the remaining samples, normalization of EpoR values were tested with each of the reference genes and assessed the extent to which relative quantification values might be influenced by RNA abundance/integrity were assessed. Specifically, a phenomenon was reported by Cronin in which greater age of formalin-fixed paraffin-embedded blocks (and the lower RNA abundance and integrity) was associated with higher relative quantification values even after normalization. Cronin et al., 164(1) Am. J. Pathol. 35-42 (2004). This effect was attributed to differential degradation of target versus endogenous control gene transcripts and was reduced by minimizing the size and range of target and control gene assay amplicon sizes. In the present data set, higher reference gene Ct values (less RNA abundance/integrity) were indeed associated with higher relative EpoR quantification upon normalization with the control gene Ppia as evidenced by the strong positive correlation between Ppia Ct values and normalized EpoR relative quantification values (FIG. 6). Similar results were obtained for normalization with the control genes Gapdh, Rplp0, Ipo8, or Tfrc. Although the pattern of normalized EpoR expression within subgroups was similar regardless of the control gene used for normalization, the overall systematic effect of RNA abundance on normalization would have precluded a comparison of EpoR levels among all patients, or would have limited comparisons to subgroups with similar amounts of RNA abundance/integrity. This systematic effect was alleviated, however, upon normalization to Hmbs, which had the shortest amplicon size among all reference gene assays tested (64 bp) (FIG. 1D).

Overall, EpoR transcript levels were determined in 106 archival formalin-fixed paraffin-embedded head and neck cancer samples for which sufficient RNA was available. All samples were among 154 cases from the ENHANCE study previously examined for EpoR protein expression using the C20 antibody (Henke et al., 2006). The range of EpoR relative quantification values for these tumors is shown in FIG. 1D. As described above, five samples with low RNA abundance produced EpoR relative quantification values of greater than mean plus 1 standard deviation and were removed, leaving a total of 101 patients for subsequent analyses.

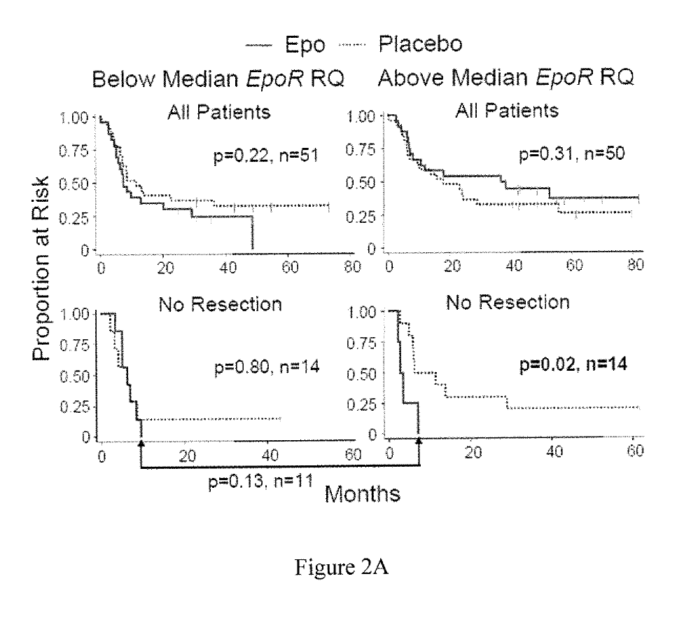

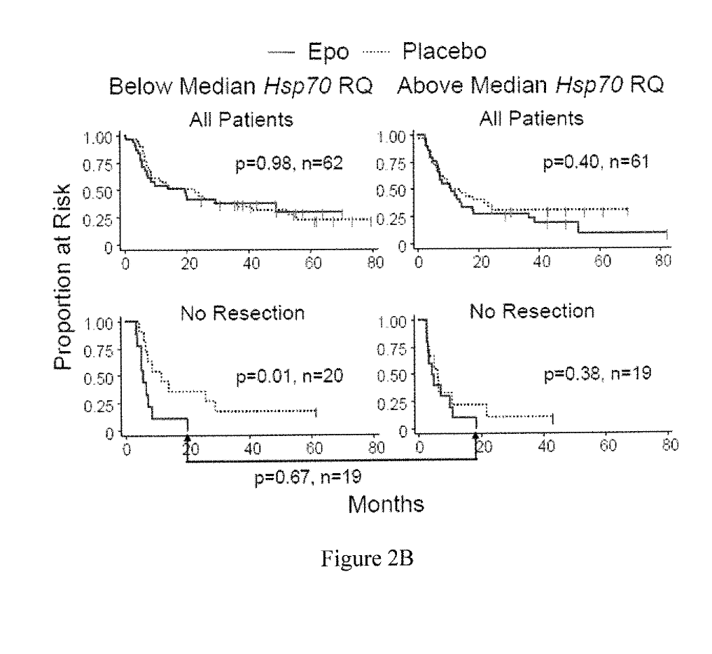

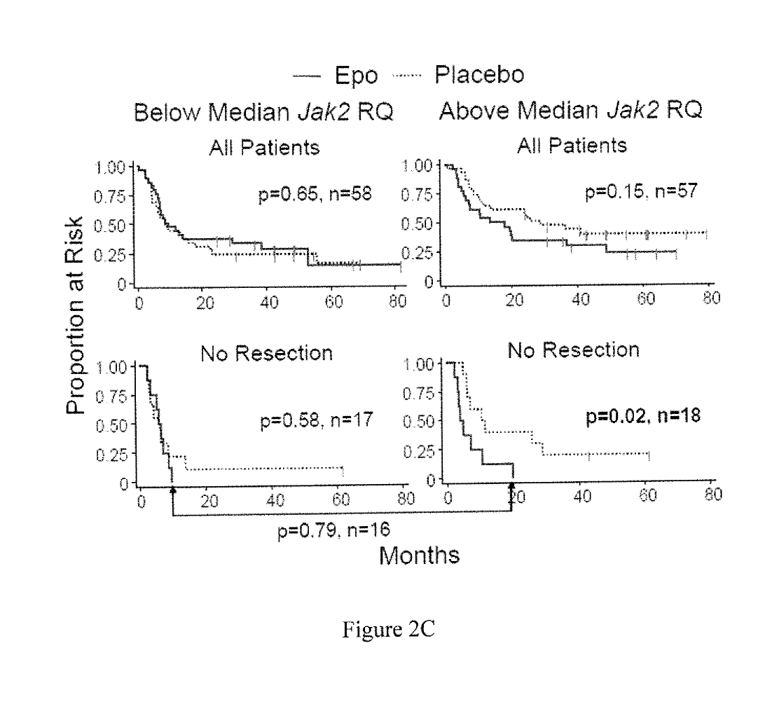

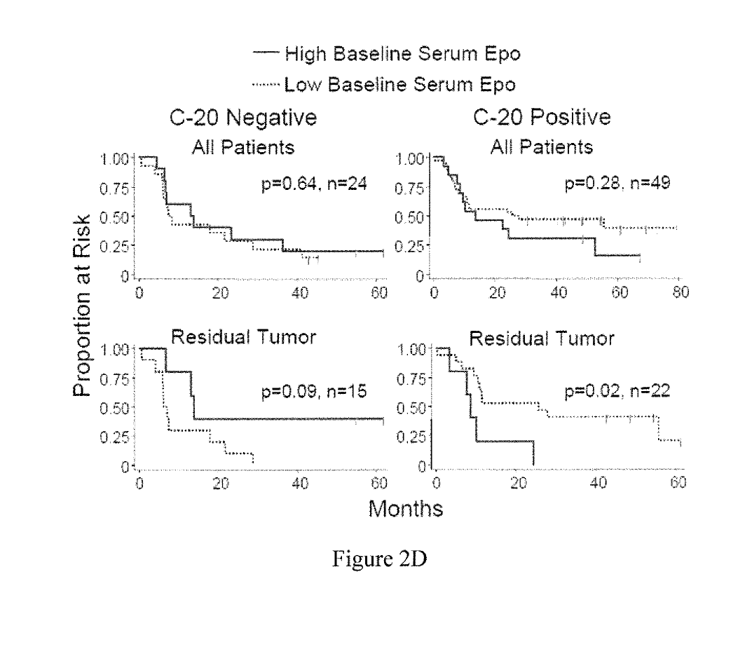

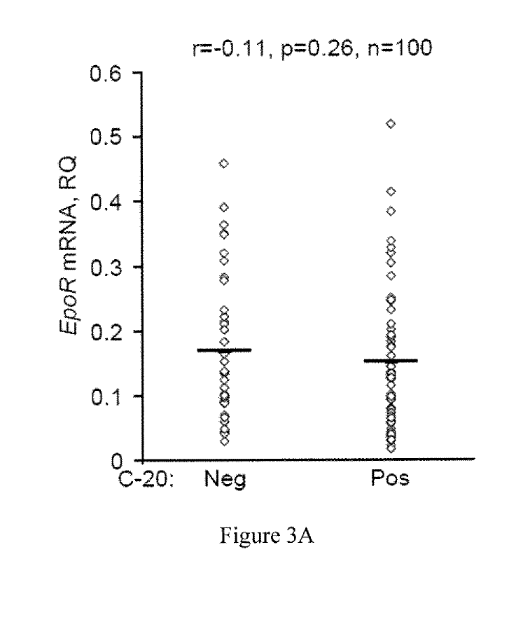

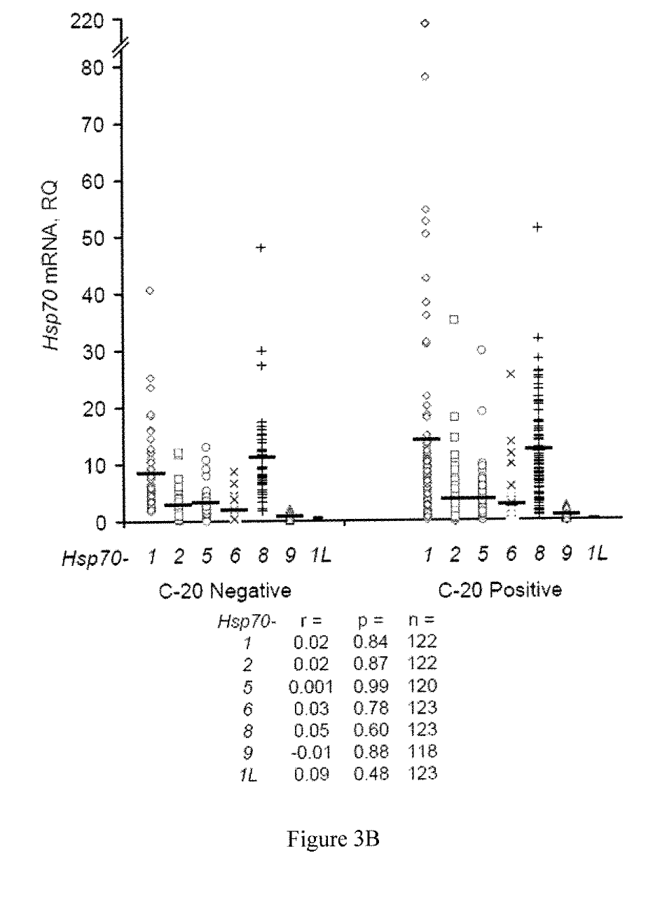

As detailed above, the primary endpoint of the ENHANCE trial was locoregional progression-free survival which was defined as the time to local tumor progression/recurrence or death. Locoregional progression-free survival was evaluated in each resection stratum in patients with tumor EpoR mRNA levels above the median versus those with EpoR levels below the median (determined separately for each stratum). In the no resection stratum, EpoR expression above the median was significantly associated with unfavorable locoregional progression-free survival in response to Epo compared to placebo (p=0.02, n=14), an effect not observed in patients with tumor EpoR levels below the median (FIG. 2A, bottom panels). Epo-treated patients with above-median EpoR levels had slightly less favorable locoregional progression-free survival as opposed to those with above-median EpoR level. 0This was not significant, however (log rank p=0.13, n=11) (FIG. 2A, bracket beneath the bottom panels). EpoR mRNA was not significantly associated with outcomes when analyzing all patients together or in the complete resection or incomplete resection strata, as shown in Table 4. There was no correlation between EpoR mRNA levels and C20 status (r=−0.11, p=0.26, n=100) (FIG. 3A).

C20 co-recognizes a motif present in all eight members of the Hsp70 family that is also contained within the twenty amino acid immunizing EpoR peptide (Elliott et al., 2006). Therefore, the extent to which Hsp70 mRNA levels correlated with C20 staining was assessed by measuring transcripts for all eight Hsp70 family members in 124 tumor samples from ENHANCE, including all 101 tumors with results for EpoR. Overall, there was no significant correlation between Hsp70 family member expression and C20 staining. A trend towards higher Hsp70-1 transcript levels in the C20-positive category was not significant (FIG. 3B). Above median Hsp70 mRNA levels were not significantly associated with Epo-dependent differences in locoregional progression-free survival in any of the resection groups (Table 4). Especially surprising was a consistent association in the no resection group between below-median levels of Hsp70 family member transcripts and an Epo associated reduction in locoregional progression-free survival (Table 4 and FIG. 2B).

Whether elevated levels of Csf2rb mRNA were associated with adverse effects of Epo was also tested. Above median Csf2rb mRNA levels were not associated with Epo-associated locoregional progression-free survival outcomes (Table 4). Finally, whether an intracellular signal transducing component of the EpoR called Jak2 was associated with adverse effects of Epo was examined. Interestingly, in the no resection stratum, Jak2 expression above the median was significantly associated with unfavorable locoregional progression-free survival in response to Epo compared to placebo, an effect not observed in patients with tumor Jak2 levels below the median (Table 4 and FIG. 2C). Among all patients with above-median Jak2, there was a trend towards inferior locoregional progression-free survival in response to Epo vs. placebo that did not reach statistical significance (Table 4 and FIG. 2C).

### Example 3

### Immunohistochemistry with C20

Formalin-fixed paraffin-embedded tissue sections obtained before treatment were assayed for EpoR protein expression retrospectively. Histopathologic diagnosis of squamous cell carcinoma of the samples was confirmed before immunohistochemical processing. A DAKO (Carpinteria, Calif.) Autostainer with ChemMate Detection Kit DAKO 5005 was used for immunohistochemistry. Following deparaffinization with xylol, alcohol, and rehydration, slides were reacted for 30 min with target retrieval solution (DAKO S1699; pH 6). Endogenous biotin was blocked using the DAKO biotin blocking system. Slides were then incubated for 30 min with a polyclonal rabbit-anti-human that recognizes human EpoR (1:200 dilution; C20; Santa Cruz Biotech., Santa Cruz, Calif.) but also cross-reacts with Hsp70 (Elliot et al., 2006). Thereafter, biotinylated goat anti-rabbit immunoglobulin was applied for 15 min, followed by alkaline phosphatase/streptavidin for another 15 minutes. Slides were developed with alkaline phosphatase/fast red and counterstained with hemalaun for 6 minutes. Fetal kidney sections served as positive control and the primary antibody was omitted for negative controls, respectively. Additionally, staining of endothelial or basal cells of the mucous membranes in individual sections was used as internal control.

Two independent reviewers unaware of all clinical data performed tissue processing and scoring of the slides. Differences between the two investigators were resolved by consensus. A four-grade scale was applied to evaluate semiquantitatively the expression intensity and proportion of positive-staining cells on the entire tissue section. Cytoplasmic or membrane staining was considered positive. Missing staining of cancer cells was considered as score 0. Score 1 showed barely, score 2, moderate, and score 3, strong cellular staining. Any positive reaction required at least 10% of cancer cells to stain. For further analyses scores 0 and 1 were regarded as negative and scores 2 and 3 as positive.

Based on the results of immunohistochemistry screening, patients were divided into two groups of patients: receptor positive and receptor negative. For the most part, characteristics of patients assigned to treatment with epoetin beta were similar to those assigned placebo. There was some imbalance in regards to resection stratum. For receptor-positive patients, there were more high-risk patients (radiation treatment without surgery) on the epoetin beta arm, while for receptor negative patients, more high-risk patients were on the placebo arm. Because the analysis stratified on resection status, however, this did not confound the results.

Consistent with the analysis of the entire trial, in the subset of patients enrolled from the Freiburg center, treatment with epoetin beta was associated with decreased locoregional progression-free survival (adjusted relative risk, 1.58; P=0.02). The negative impact of epoetin beta appears to be restricted to patients whose cancers expressed EpoR. The resection status adjusted relative risks for loco-regional failure or death were 2.07 (p<0.01) for receptor positive patients and 0.94 (p=0.86) for receptor negative patients (treatment group v placebo). Note that any imbalances between treatment and placebo in resection status do not contribute to these relative risks as they have been adjusted for through stratification.

### Example 4

### Evaluating the Effects of Endogenous Epo Produced by the Body

Another way of evaluating whether Epo can stimulate tumor progression is to examine whether elevated levels of endogenous Epo levels correlate with poor outcome, using patients assigned to the placebo group. The ENHANCE study documented pre-treatment serum Epo levels prior to randomization. Whether locoregional progression-free survival was associated with elevated baseline serum Epo levels, tumor C20 status, or EpoR, Hsp70, and Jak2 transcript levels were evaluated. Among all placebo treated patients, outcomes in patients with high versus low serum Epo levels were not significantly different when stratifying for C20 status (FIG. 2D, top panels). In patients with unresected or incompletely resected tumors, however, elevated endogenous Epo levels were associated with significantly impaired locoregional progression-free survival if tumors were C20 positive (p=0.02, n=22), and improved locoregional progression-free survival if tumors were C20 negative (p=0.09, n=15) (FIG. 2D). In contrast, locoregional progression-free survival did not significantly differ between patients with high versus low serum Epo levels when stratifying for EpoR, Hsp70, or Jak2 mRNA levels (see Table 5).

1. A method for determining the risk that a cancer patient treated with an erythropoiesis-stimulating agent (ESA) may experience tumor progression due to ESA treatment, comprising the steps of:
(a) obtaining a tumor sample from said patient;
(b) determining the quantity of EpoR, Jak2, and/or Hsp70 expression in said sample; wherein an increase in the expression level of said EpoR, Jak2, and/or a decrease in the expression level of Hsp70 in said sample as compared with control-level gene expression is indicative of increased risk of tumor progression upon treatment with an ESA.

2. (canceled)

3. (canceled)

4. A diagnosis kit for detecting biomarkers associated with ESA-induced tumor progression, comprising: at least one container means for accepting a tumor sample; at least one reagent for carrying out PCR amplification; reagents for carrying out RNA extraction, cDNA synthesis, and pre-amplification; and primers for determining EpoR, Jak2, and/or Hsp70 mRNA and control gene mRNA levels.

5. A method for determining the risk that a cancer patient treated with an erythropoiesis-stimulating agent (ESA) may experience tumor progression due to ESA treatment comprising the steps of:
(a) obtaining a tumor sample from said patient;
(b) detecting the presence of Epo receptor (EpoR) in said sample; wherein increased presence of EpoR in said sample as compared to control levels is indicative of increased risk of tumor progression upon treatment with an ESA.

6. (canceled)

7. (canceled)

8. The method of claim 1, wherein said determining step is done by PCR analysis.

9. The method of claim 8, wherein said PCR analysis is automated.

10. The method of claim 5, wherein said detecting is done by measuring the quantitative binding of said EpoR with erythropoietin (Epo).

11. The method of claim 5, wherein the detecting is done by measuring the quantitative binding of anti-EpoR antibody to said EpoR.

12. The method of claim 11, wherein said measuring is by ELISA.

13. The method of claim 12, wherein said ELISA is automated.
